(19) United States
(12) Patent Application Publication
(10) Pub. No.: US 2008/0096951 A1
(43) Pub. Date: Apr. 24, 2008

(54) Carvedilol Monocitrate Monohydrate

(75) Inventors: Wei CHEN, Collegeville, PA (US); Choon K. Oh, Collegeville, PA (US); Li-Jen J. Ping, King of Prussia, PA (US); Paul G. Spoors, King of Prussia, PA (US)

Correspondence Address: GLAXOSMITHKLINE;Corporate Intellectual Property - UW2220, P.O. Box 1539, King of Prussia, PA, 19406-0939, US

(73) Assignee: SB Pharmo Puerto Rico Inc.

(21) Appl. No.: 11/962,163

(22) Filed: Dec. 21, 2007

Related U.S. Application Data
(63) Continuation of application No. 10/512,628, filed on Oct. 26, 2004.
(60) Provisional application No. 60/376,599, filed on Apr. 30, 2002.
(60) Provisional application No. 60/383,287, filed on May 23, 2002.

(51) Int. Cl. A61K 31/40; A61P 9/00; C07D 209/82

(52) U.S. Cl. 514/411; 548/441

(57) ABSTRACT

This invention relates to carvedilol monocitrate monohydrate, compositions containing this salt of carvedilol and methods of using this compound to treat hypertension, congestive heart failure and angina.

### CROSS REFERENCE TO PRIOR APPLICATIONS

This application is a continuation of U.S. application Ser. No. 10/512,628 filed 26 Oct. 2004 now pending which is a 371 application of PCT/US2003/013832 filed 30 Apr. 2003 which claims the benefit of U.S. Provisionals 60/376,599 filed 30 Apr. 2002 and 60/383,287 filed 23 May 2002.

### FIELD OF THE INVENTION

This invention relates to a salt of carvedilol, compositions containing this compound and methods of using the compound in the treatment of certain disease states in mammals, in particular man. More specifically, the present invention relates to a novel crystalline form of carvedilol monocitrate monohydrate, which is the monocitrate salt of 1-(carbazol-4-yloxy-3-[[2-(o-methoxyphenoxy)ethyl]amino]-2-propanol, compositions containing this compound, and methods of using carvedilol monocitrate monohydrate to treat hypertension, congestive heart failure and angina.

### BACKGROUND OF THE INVENTION

The compound, 1-(Carbazol-4-yloxy-3-[[2-(o-methoxyphenoxy)ethyl]-amino]-2-propanol is known as carvedilol. This compound has the following structure: 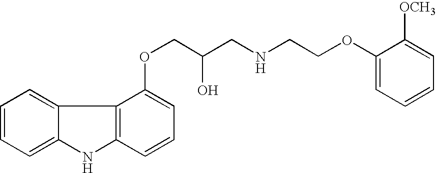
and is claimed in U.S. Pat. No. 4,503,067 (assigned to Boehringer Mannheim, GmbH, Mannheim-Waldhof, Fed. Rep. of Germany), issued Mar. 5, 1985.

Carvedilol is currently synthesized as free base for incorporation in medication that is available commercially. It is a racemic mixture of the R(+) and S(−) enantiomers, where nonselective β-adrenoreceptor blocking activity is present in the S(−) enantiomer and α-adrenergic blocking activity is present in both R(+) and S(−) enantiomers. This unique feature contributes to the two complementary pharmacologic actions: mixed venous and arterial vasodilation and non-cardioselective, beta-adrenergic blockade.

Carvedilol is used for treatment of hypertension, congestive heart failure and angina. The currently available product is a conventional, tablet prescribed as a twice-a-day medication in the United States.

Carvedilol contains an α-hydroxyl secondary amine, with a pKa of 7.8. It exhibits predictable solubility behaviour in neutral or alkaline media, i.e. above pH 9.0, the solubility is relatively low (<1 μg/mL). Its solubility increases with decreasing pH and reaches a plateau near pH 5: i.e. saturation solubility is ca 23 μg/mL at pH 7 and ca 100 μg/mL at pH 5 at room temperature. At lower pH values (pH 1 to 4 in buffer systems), solubility is limited by the solubility of the protonated form of carvedilol or its salt formed in-situ. The hydrochloride salt generated in-situ in an acidic medium, such as simulated gastric fluid, is less soluble in this medium than the protonated carvedilol itself.

Additionally, the presence of the α-hydroxyl secondary amine group confers the propensity to chemically react with excipients normally included in a dosage form to aid manufacture, maintain quality or enhance dissolution rate. For example, this type of amine groups can react with aldehydes or ester functional groups through nucleophilic reactions. Many excipients have ester functional groups. Aldehydes and other such residues are common residues in excipients. This often results in marginal or unacceptable chemical stability upon storage.

A salt form with greater aqueous solubility, allied to greater chemical stability offers potential benefit for the provision of medicinal products containing this drug, in particular those where it is desired to prolong drug levels in the systemic system by sustaining absorption along the gastro-intestinal tract, particularly regions of neutral pH where carvedilol solubility is minimal.

Surprisingly, it has now been shown that a novel crystalline form of carvedilol monocitrate salt, can be isolated as pure, crystalline solid that exhibits much higher aqueous solubility than the corresponding free base or other prepared crystalline salts such as the hydrochloride salt. It also has the potential for improved stability of carvedilol in formulations given that the secondary amine, a moiety that is pivotal to degradation processes is protonated as a salt.

### BRIEF DESCRIPTION OF THE FIGURES

FIG. 1 is an FT-IR spectrum for carvedilol monocitrate monohydrate.

FIG. 2 is an X-Ray Powder Diffractogram (XRPD) for carvedilol monocitrate monohydrate.

### SUMMARY OF THE INVENTION

The present invention provides a novel crystalline form of carvedilol, namely carvedilol monocitrate monohydrate.

The present invention also provides pharmaceutical compositions containing carvedilol monocitrate monohydrate and the use of this compound in the treatment of hypertension, congestive heart failure and angina.

### DETAILED DESCRIPTION OF THE INVENTION

In accordance with the present invention, it has been unexpectedly found that carvedilol monocitrate monohydrate can be readily isolated as a novel crystalline form, which displays much higher solubility when compared to the free base of carvedilol.

Carvedilol is claimed in U.S. Pat. No. 4,503,067 (the '067 patent). Reference should be made to said patent for its full disclosure, including the methods of preparing and using this compound. The entire disclosure of the '067 patent is incorporated herein by reference.

The crystalline carvedilol citrate salt of the instant invention can be prepared by making an aqueous citric acid solution saturated with carvedilol, either by lowering the temperature of the solution, or slowly evaporating water from the solution. In addition, it can be prepared by crystallization from an acetone-water solvent system containing carvedilol and citric acid.

A particularly useful and surprising attribute of the instant crystalline form of carvedilol concerns the capability to provide both R(+) and S(−) forms in an enantiomeric 1 to 1 ratio, although the citrate counter ion in this salt form is chiral. This avoids generation of yet more optically active forms that could potentially complicate stability, dissolution rates, in vivo absorption metabolism and possibly pharmacologic effects.

Such properties indicate that it may be particularly suitable for inclusion in medicinal agents. Its solubility may facilitate provision of a dosage form from which the drug substance becomes available for bioabsorption throughout the gastrointestinal tract, in particular the lower small intestine and colon. Hence, it may be possible to develop stable controlled release dosage forms for once-per-day dosage, delayed release or pulsatile release to optimize therapy by matching pharmacokinetic performance with pharmacodynamic requirements.

Thus, this invention also relates to a pharmaceutical composition comprising an effective amount of carvedilol monocitrate monohydrate with any of the characteristics noted herein, in association with one or more non-toxic pharmaceutically acceptable carriers and/or diluents thereof, and if desired, other active ingredients. The compositions are prepared using conventional techniques, such as mixing, blending and the like. The compositions may be administered orally, intravascularly, intraperitoneally, subcutaneously, intramuscularly or topically. Preferably, the composition is adapted for oral administration. The composition is presented as a unit dose. Such a composition is taken preferably from 1 to 2 times daily, most preferably once daily. The preferred unit dosage forms include tablets or capsules. Typically, the oral maintenance dose is between about 25 mg and about 50 mg, preferably given once daily.

This invention further relates to the use for treatment of hypertension, congestive heart failure and angina in a mammal in need thereof, which method comprises administering to said mammal an effective amount of carvedilol monocitrate monohydrate with any of the characteristics noted herein.

The following examples are illustrative of the instant invention. These examples are not intended to limit the scope of this invention as defined hereinabove and as claimed hereinbelow.

### EXAMPLES

### Example 1

In a 150 mL glass beaker, 100 gram of 20% w/w citric acid solution was prepared and 2.2 gram of carvedilol was added. The solution became slightly brownish after 15 minutes stirring, with only a little solid sticking on the bottom of the beaker. The beaker was then placed in a fume hood for evaporation. After staying in the hood overnight, large single crystals appeared in the beaker. The solid crystals were isolated and dried in a desiccator under vacuum. Similarly single crystals of citrate salt could be obtained by slow evaporation of carvedilol/citric acid solutions (containing citric acid 5%, 10% or 20% w/w) in Petri dishes (150 mm diameter) placed in a desiccator connected to a house vacuum.

### Example 2

A 250 mL three-necked flask equipped with stirrer bar, thermometer, and an addition funnel is charged with acetone (20 mL, 2.5 volumes). The solution is sequentially charged with carvedilol (8 g, 19.7 mmol), and 2 M citric acid solution (40 mL, 5 volumes). Upon addition of the citric acid solution, the slurry dissolves quickly. The solution is filtered through a Buchner funnel fitted with Whatman filter paper and the solution is returned to a 250 mL flask fitted with a stirrer. To the light brown solution is added water (20 mL, 2.5 volumes). No exotherm is noted. The reaction mixture becomes cloudy but disappears upon stirring (heating up to 40° C. maybe needed to remove cloudiness). The mixture is stirred at room temperature and when judged clear is charged with carvedilol monocitrate monohydrate seeds (80 mgs) in one portion. An immediate cloudiness is observed (solid starts to precipitate out over 12-24 hours). The precipitate formed is stirred for 24-48 hours and is filtered through a Buchner funnel fitted with Whatman filter paper and the collected cake is washed with water (2×16 mL). The cake is dried in the oven under house vacuum at 50° C. to a constant weight. The cake (7.95 g, 67%) is weighed and stored in a polyethylene container.

### Example 3

A suitable reactor is charged with acetone. The solution is sequentially charged with carvedilol, and aqueous citric acid solution. Upon addition of the citric acid solution, the slurry dissolves quickly. To the solution is added water. The mixture is stirred at room temperature and is charged with carvedilol seeds in one portion. The precipitate formed is stirred for a period of time, filtered and the collected cake is washed with water. The cake is dried under vacuum to a constant weight and stored in a polyethylene container.

### Characterization

The HPLC assay and ¹H-NMR revealed that the molar ratio of carvedilol and citric acid in carvedilol citrate salt prepared was approximately 1:1. The characterization by several other techniques are listed below:

Scanning Electron Microscopy (SEM):

The SEM used for the study was a Hitachi S-3500N. SEM was performed using an acceleration voltage of 5 kV. The samples were gold sputtered.

The carvedilol monocitrate salt consists of crystals with plate-shape, and various sizes depending on the preparation method. Crystals as large as 1 mm width and length were observed.

Differential Scanning Calorimetry (DSC)

DSC measurements were performed with a MDSC 2920 (TA Instruments, Inc.). Approximately 5 mg of the sample was placed in an open aluminum pan. The sample was scanned at 10° C./min. An endothermic event was observed with an onset temperature near 82-83° C. The heat of fusion was calculated as 63 kJ/mol.

Fourier Transform Infrared Spectroscopy (FT-IR):

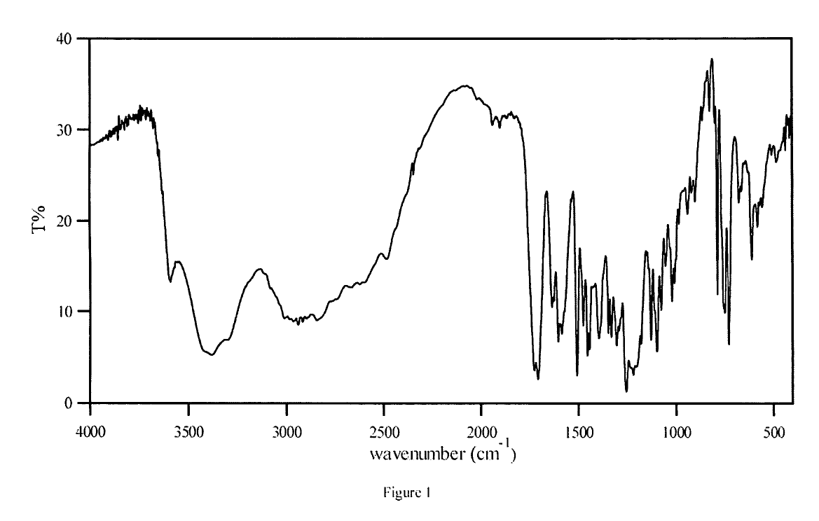

Approximately 2 mg of sample was diluted with 300 mg of dried potassium bromide (KBr). The mixture was ground with a mortar and pestle, then transferred to a die that is placed under high pressure for 3 minutes. The instrument was a PerkinElmer Spectrum GX FTIR instrument. Forty scans were collected at 4 cm⁻¹ resolution. The typical FT-IR spectrum of carvedilol monocitrate salt is shown in FIG. 1. The characteristic peaks in the 1800 to 600 cm⁻¹ region are found at about 1727, 1709, 1636, 1625, 1604, 1586, 1508, 1475, 1454, 1443, 1396, 1346, 1332, 1305, 1256, 1221, 1129, 1096, 1077, 1054, 1021, 1008, 984, 939, 919, 902, 826, 787, 755, 749, 729, 676, 664, 611 cm⁻¹.

X-Ray Powder Diffraction (XRPD):

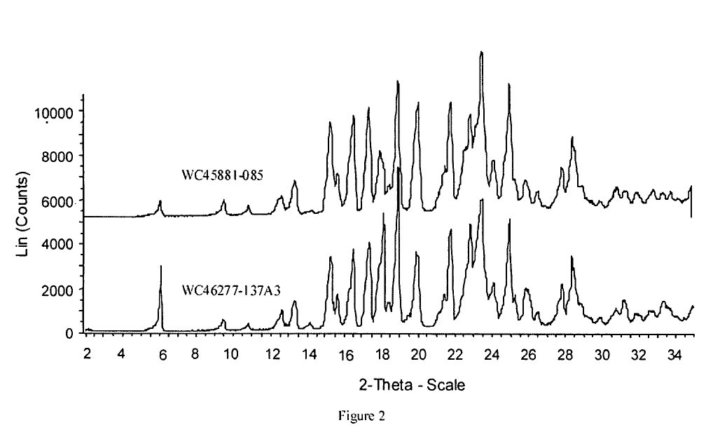

XRPD patterns were collected using a Philips X'Pert Pro Diffractometer. Approximately 30 mg of sample was gently flattened on a silicon sample holder and scanned from 2-35 degrees two-theta, at 0.02 degrees two-theta per step and a step time of 2.5 seconds. The sample was rotated at 25 rpm. The XRPD patterns of two different batches of Carvedilol monocitrate salt are shown in FIG. 2.

Solubility in Water:

Glass vials containing water and excess amount of carvedilol salts were shaken by a mechanical shaker at ambient conditions. Aliquots were taken out at various time-point, filtered through 0.45 μm Acrodisc GHP filter. The pH of the filtered solutions was measured and suitable dilution was performed prior to UV-Vis analysis of carvedilol concentration.

The solubility of carvedilol monocitrate salt in water at room temperature was determined. The drug concentrations and pH values at different time-points are presented in Table 1. This crystalline form of carvedilol monocitrate salt exhibited high solubility in water (1.63 mg/mL at 1 hour and 1.02 mg/mL at 48 hour).

TABLE 1
Aqueous Solubility (expressed as mg of carvedilol
free base/mL of solution) at 25° C. for carvedilol
free base and its monocitrate salt.

| | Carvedilol | Carvedilol |
|---|---|---|
| Time, hr | Free Base | Mono-Citrate salt |
| 1 | 0.0098 | 1.63 (pH = 3.5) |
| 4 | | 1.47 (pH = 3.4) |
| 24 | 0.0116 | 1.07 (pH = 3.0) |
| 48 | | 1.02 (pH = 3.0) |

Carvedilol monocitrate salt has two free carboxylic acid groups in one unit salt, which contributes the low pH value (near pH 3) observed for monocitrate salt when dissolved in water. This may potentially lead to improved formulations by providing a low pH microenvironment within the formulation as it traverses the GI tract, particularly concerns the lower GI tract, where the pH of the environment is near neutral pH and the solubility of the drug substance is limited. Such a pH microenvironment should lead to greater dissolution rate because of higher solubility in the solid/liquid interface, leading to improved absorption of drug in the the lower GI tract thereby prolonging absorption and, in consequence blood levels and allowing less frequent dosing. Therefore, a once-per-day carvedilol formulation may be possible by incorporating carvedilol monocitrate salt, which will be more convenient for the patients and will also result in higher patient compliance.

Crystalline Structure of Carvedilol Monocitrate Salt

The crystalline structure of carvedilol citrate salt was determined by Single Crystal X-Ray Diffraction analysis on the large crystals formed by evaporation. The result indicated that the salt form was a carvedilol monocitrate, where the molar ratio of carvedilol and citric acid was 1:1. Surprisingly, the hydroxyl of carvedilol is disordered in the crystalline packing. In other words, the monocitrate salt has both R(+) and S(−) carvedilol enantiomers at 1:1 molar ratio, and the two enantiomers are randomly distributed, without any specific order.

This crystalline packing habit is very unusual for a salt formed between a chiral compound and a chiral counter-ion (monocitrate). Typically, chiral counter-ion tends to differentiate the two stereoisomers of the compound when forming crystals. However, in the case of the monocitrate salt, there seems to be enough space in the crystal packing to allow the carbonyl group of the terminal carboxylic acid group of citrate to form equivalent hydrogen bond with the hydroxyl from either the R(+) or the S(−) carvedilol stereoisomer.

This avoids generation of yet more optically active forms that could potentially complicate stability, dissolution rates and possibly in vivo absorption and pharmacologic effects.

The above data demonstrates that a novel crystalline form of carvedilol monocitrate monohydrate can be prepared with a unique crystalline packing habit, which exhibits high aqueous solubility and can provide a low pH microenvironment for enhanced dissolution.

It is to be understood that the invention is not limited to the embodiments illustrated hereinabove and the right is reserved to the illustrated embodiments and all modifications coming within the scope of the following claims.

The various references to journals, patents, and other publications which are cited herein comprise the state of the art and are incorporated herein by reference as though fully set forth.

What is claimed is:

1. A compound which is crystalline carvedilol monocitrate monohydrate.

2. The compound according to claim 1 having an infrared spectrum which comprises characteristic absorption bands expressed in wave numbers as shown in FIG. 1.

3. The compound according to claim 2 having characteristic peaks in the 1800 to 600 cm⁻¹ region at about 1727, 1709, 1636, 1625, 1604, 1586, 1508, 1475, 1454, 1443, 1396, 1346, 1332, 1305, 1256, 1221, 1129, 1096, 1077, 1054, 1021, 1008, 984, 939, 919, 902, 826, 787, 755, 749, 729, 676, 664, 611 cm⁻¹.

4. The compound according to claim 1 having an X-ray powder diffraction pattern which comprises characteristic peaks as shown in FIG. 2.

5. A pharmaceutical composition comprising the compound according to claim 1 and a pharmaceutically acceptable carrier.

6. A method of treating hypertension, congestive heart failure or angina which comprises administering to a subject in need thereof an effective amount of the compound according to claim 1.

7. A process for preparing carvedilol monocitrate monohydrate which comprises making an aqueous citric acid solution saturated with carvedilol.

8. A process for preparing carvedilol monocitrate monohydrate which comprises crystallization from an acetone-water solvent system containing carvedilol and citric acid.
